(19) United States
(12) Patent Application Publication
(10) Pub. No.: US 2025/0009638 A1
(43) Pub. Date: Jan. 9, 2025

(54) FILM FORMING COSMETIC INGREDIENTS COMPRISING BOSWELLIA THICK OIL AND OAT KERNEL OIL

(71) Applicant: GIVAUDAN SA, VERNIER (CH)

(72) Inventors: Juliette LOYENET, Pomacle (FR); Celine DINANT-BILINSKI, Pomacle (FR); Yohan ROLLAND, Avignon Cedex 09 (FR)

(21) Appl. No.: 18/712,609

(22) PCT Filed: Dec. 7, 2022
(86) PCT No.: PCT/EP2022/084741

§ 371 Date: May 22, 2024

(30) Foreign Application Priority Data
Dec. 10, 2021 (GB) 2117863.7

(51) Int. Cl. A61K 8/92; A61Q 5/00; A61Q 17/04

(52) U.S. Cl. CPC A61K 8/922; A61Q 5/00; A61Q 17/04

(57) ABSTRACT

A cosmetic ingredient comprising Boswellia thick oil (Boswellia serrata oil) and oat kernel oil (Avena Sativa (Oat) Kernel oil) is provided, as well as cosmetic compositions comprising the same and methods of use thereof.

The invention relates to a cosmetic ingredient comprising Boswellia thick oil (Boswellia serrata oil) and oat kernel oil (Avena Sativa (Oat) Kernel oil), to cosmetic compositions comprising the same, and to methods of use thereof.

The need for cosmetic ingredients for imparting water resistance and aiding retention of cosmetic actives in personal care compositions is well known. Without them, personal care actives may wash off, wear off, be re-emulsified, or otherwise lose their efficacy. Water resistance properties are particularly important for sun care compositions in order to prevent the protective composition from being easily removed from a keratinous substrate surface by sweat and exposure to water.

It is further important that the water resistance imparting ingredients are not tacky and do not impart a bad aesthetic feel to consumers when formulated into leave-on formulations, as aesthetics is one of the most important considerations in a consumer's selection of, or at least loyalty to, a personal care composition.

U.S. Pat. No. 4,978,527 teaches a film-forming emulsion containing iodine and methods of use. The film-forming emulsion comprises: (a) a substantially water resistant film-forming copolymer phase comprising A, B and C monomers wherein A is a “soft” monomer wherein the corresponding homopolymer has a glass transition temperature (Tg) of less than about −15° C., and is present as about 15 to 80% of the total weight of all monomers in the copolymer, B is a “hard” monomer wherein the corresponding homopolymer has a Tg of more than about −5° C., and is present as about 20 to 70% of the total weight of all monomers in the copolymer, and C is a monomer capable of complexing iodine and delivering it to the skin and is present as about 1 to 15% of the total weight of all monomers in the copolymer; (b) about 0.05 to 15% of iodine based on total emulsion weight; (c) an effective amount of an emulsifying agent; and (d) about 30 to 95% by weight of water.

U.S. Pat. No. 4,584,192 teaches a film-forming composition containing an antimicrobial agent and methods of use. The film-forming composition comprises (a) a film-forming copolymer consisting essentially of copolymerized A, B, and C monomers as follows: A is a monomeric acrylic or methacrylic acid ester of an alkyl alcohol containing a single hydroxyl, the alcohol being further described as having from 2 to about 14 carbon atoms when the A monomer is an acrylic acid ester, and about 7 to 18 carbon atoms when the A monomer is a methacrylic acid ester, the amount by weight of A monomer being about 15 to 80% of the total weight of all monomers in the copolymer; B is a monomeric methacrylic acid ester of an alkyl alcohol containing a single hydroxyl, the alcohol being further described as having from 1 to about 6 carbon atoms, the amount by weight of B monomer being about 20 to 70% of the total weight of all monomers in the copolymer; and C is an N-vinyl lactam, the amount by weight of which being about 1 to 15% of the total weight of all monomers in the copolymer; (b) an effective amount of a broad spectrum antimicrobial agent; the composition being dermatologically acceptable, and, when applied to skin from a fugitive solvent, being capable of forming a clear, substantially fluid-resistant, substantially tack-free, flexible film which adheres to skin and releases the antimicrobial agent to skin.

WO 2013/148614 teaches water resistant polymers for personal care. The personal care compositions comprise a polymer comprising: (a) one or more polymer comprising, as polymerized units, (i) 75% to 35% by weight, based on the weight of said polymer, one or more (meth)acrylate monomer selected from at least one of C₁-C₄ (meth)acrylate, (meth)acrylic acid, styrene, or substituted styrene, and (ii) 25% to 65% by weight, based on the weight of said polymer, one or more hydrophobic monomer, including hydrophobically substituted (meth)acrylate monomers, with alkyl chain length from C₈ to C₂₂ and, (iii) optionally a crosslinker, and, (b) at least one personal care active, other than a sun care active.

WO 2015/153461 teaches vinyl lactam-derived polymers, compositions thereof having enhanced water resistance, and methods of use thereof. The polymer comprises repeating units derived from (a) from 16% by weight to about 35% by weight of said polymer of at least one N-vinyl lactam monomer; (b) at least one monomer selected from the group consisting of functionalized and unfunctionalized C₁-C₆ alkyl (meth)acrylates, C₁-C₆ alkyl (meth)acrylamides and combinations thereof; and (c) at least one monomer selected from the group consisting of functionalized and unfunctionalized C₈-C₃₀ branched alkyl (meth)acrylates, C₈-C₃₀ branched alkyl (meth)acrylamides, and combinations thereof; wherein the polymer has a glass transition temperature of greater than about 45° C.

WO 2017/087645 teaches non-aqueous compositions of polymers derived from monomers having an acryloyl moiety and a lactam moiety and applications thereof. The non-aqueous composition comprises a film forming polymer comprising repeating units derived from at least: (a) at least one monomer comprising at least one functionalized or unfunctionalized acryloyl moiety and at least one lactam moiety; (b) at least one monomer selected from the group consisting of functionalized and unfunctionalized C₁-C₇ alkyl (alk)acrylates, C₁-C₇ alkyl (alk)acrylamides, and combinations thereof; and (c) at least one monomer selected from the group consisting of functionalized and unfunctionalized C₈-C₆₀ alkyl (alk)acrylates, C₈-C₆₀ alkyl alkyl (alk)acrylamides, and combinations thereof.

As can be seen from the above, most of the water resistance imparting ingredients known today are synthetic. However, consumers are more and more aware of the environment and are looking for natural alternatives.

Accordingly, what is needed is a water resistance imparting ingredient of natural origin, which possesses excellent retention of active ingredients when water is present and provides a good aesthetic performance.

Surprisingly, it was found that the cosmetic ingredient of the invention provides, among many other benefits, the important benefits of enhanced water-resistance and good skin feel. The cosmetic ingredient may be formulated into a wide variety of compositions for many important applications, in particular for cosmetic applications such as sun care.

In a first aspect, the present invention provides a cosmetic ingredient comprising Boswellia thick oil and oat kernel oil in a ratio of between 60:40 and 15:85 by weight.

In a second aspect, the present invention provides a cosmetic composition comprising the cosmetic ingredient and a cosmetic active.

In a third aspect, the present invention provides a method of improving the water resistance of a cosmetic composition, in particular of a sun protection skin or hair care composition, comprising the step of incorporating the cosmetic ingredient in the cosmetic composition.

In a fourth aspect, the present invention relates to the use of the cosmetic ingredient as a film forming agent.

In a fifth aspect, the present invention relates to the use of the cosmetic ingredient to improve the water resistance of a cosmetic composition.

The invention will be explained in more detail below.

The cosmetic ingredient of the invention comprises Boswellia thick oil (Boswellia serrata oil) and oat kernel oil (Avena Sativa (Oat) Kernel oil).

Throughout this application, the terms “Boswellia thick oil”, “Boswellia resin” and “Boswellia serrata oil” are used interchangeably.

Throughout this application, the terms “oat kernel oil”, “oat oil”, “Avena Sativa Kernel oil” and “Avena Sativa (Oat) Kernel oil” are used interchangeably.

Boswellia is a genus of trees in the order Sapindales, known for its fragrant resin. Boswellia species are moderate-sized flowering plants, including both trees and shrubs, and are native to tropical regions of Africa and Asia. Boswellia serrata is a plant that produces Indian frankincense. The plant is native to much of India and the Punjab region that extends into Pakistan.

Traditionally, Boswellia is extracted with hexane. For example, the process may comprise one or more of the following steps: extraction of Boswellia gum resin with hexane; removal of solvent to obtain Boswellia crude oil; extraction of the crude oil with aqueous ethanol to obtain an ethanolic extract; removal of solvent (optionally under vacuum) to obtain deodorized Boswellia thick oil. Optionally, additional steps of deodorization and/or color reduction may be performed, for instance by distillation under vacuum, possibly with a co-solvent, use of activated charcoal of any form, or any other such steps well known to the skilled person.

However, Indfrag Biosciences has recently succeeded in preparing Boswellia thick oil by extraction with alcohol, providing a fully natural product. For example, the process may comprise one or more of the following steps: extraction of dried Boswellia serrata gum with ethanol to obtain an ethanolic extract; precipitation of terpenic acid with caustic; filtration; concentration.

Boswellia thick oil is commercially available from e.g. Indfrag Biosciences. Typically, it is a thick liquid with a yellow to dark brown color, a specific gravity of about 0.99 to 1.2, a refractive index of about 1.48 to 1.55, a water content of ≤2%, and a peroxide value of <10 meq O₂/kg. However, slightly lower or higher values, e.g. for the specific gravity, are also acceptable. Indfrag's Boswellia thick oil is 100% natural according to the ISA 16128 standard.

Boswellia thick oil is not to be confused with Boswellia (or frankincense) essential oil: The oleo gum-resins of Boswellia typically contain about 60-70% of ethanol-soluble Boswellia resin, about 25-30% of a water-soluble fraction, and about 5-10% of Boswellia essential oils.

Boswellia thick oil or Boswellia resin is mainly composed of mono-, di- and tri-terpenic acids. The main family (triterpenic acids) typically contains the following six main boswellic acids:
about 15-25% of 3-O-acetyl-11-keto-β-boswellic acid (AKBA)
about 15-25% of 3-O-acetyl-β-boswellic acid (β-ABA)
about 15-25% of β-boswellic acid (β-BA)
about 10-20% of α-boswellic acid (α-BA)
about 5-15% of 3-O-acetyl-α-boswellic acid (α-ABA)
about 1-10% of 11-keto-β-boswellic acid (KBA)

Frankincense essential oil, on the other hand, contains as major components limonene and α-pinene. A typical composition of Boswellia essential oil consists of about 45.1% of α-pinene, 14.9% of limonene, 10.6% of α-thuyene, 8.3% of myrcene, 7.6% of sabinene, 3.4% of para-cymene, and 10.1% of other components.

And the water-soluble fraction typically is a polysaccharidic fraction with different types of proteoglycans and glycoproteins (classical arabinogalactan proteins).

The oat (Avena sativa), sometimes called the common oat, is a species of cereal grain grown for its seed. While oats are suitable for human consumption as oatmeal and rolled oats, one of the most common uses is as livestock feed. Oats have also been used for thousands of years as a way to soothe and treat the skin topically. The use of oats and the extracts made from them first became popular as a cosmetic ingredient during the 1930s when their power to relieve itching and protect the skin became widely known.

Oat kernel oil is typically produced by extraction of dehulled and heat-treated oat kernels.

For example, the oat kernels may be milled and defatted by water/ethanol extraction to obtain a crude oil and a flour rich in protein and beta-glucan. Additional fractionation may be done on the crude oil by a refining process based on polar lipid content, e.g. into a fraction with about 4% of polar lipids, one with about 15% of polar lipids, and one with about 40% of polar lipids (indicative values). Optionally, additional steps of deodorization and/or color reduction may be performed, for instance by distillation under vacuum, possibly with a co-solvent, use of activated charcoal of any form, or any other such steps well known to the skilled person.

Oat kernel oil is commercially available from e.g. Oat Cosmetics. Typically, it is a yellowish-brown liquid with a density of about 1.0 to 1.1 kg/l, a water content of less than 20,000 ppm (preferably less than 1500 ppm) and a peroxide value of <10 meq O₂/kg fat. However, slightly lower or higher values, e.g. for the density, are also acceptable.

Both oils are fully natural products compliant with several international standards, such as COSMOS and ISO 16128.

Surprisingly, it has been found that by providing Boswellia thick oil and oat kernel oil in a ratio of between 60:40 and 15:85 by weight, a superior water resistance imparting effect can be achieved compared to either oil alone or in other ratios.

The cosmetic ingredient of the invention shows pleasant aesthetic feel, great film formation, and has excellent retention of active ingredients in the presence of water.

In an embodiment, the cosmetic ingredient of the invention comprises Boswellia thick oil and oat kernel oil in a ratio of between 50:50 and 20:80 by weight, more preferably of between 45:55 and 25:75 by weight, even more preferably of between 45:55 and 35:65 by weight, most preferably of about 40:60 by weight. It has been found that, depending on the application, these ratios are preferred and provide a superior water resistance.

The cosmetic ingredient of the invention may comprise other materials, or it may essentially or completely consist of Boswellia thick oil and oat kernel oil. In an embodiment, the cosmetic ingredient consists of Boswellia thick oil and oat kernel oil.

Crude oat oil typically has a polar lipid content of about 8-25% of the total lipids, depending on the extraction solvent used.

It has been found that oat kernel oil with a relatively high polar lipid content provides particularly good results.

Therefore, in an embodiment, the oat kernel oil has a polar lipid content of at least about 25% by weight of the oil, more preferably of at least about 30% by weight, still more preferably of at least about 35% by weight, and most preferably of about 40% by weight. The oat kernel oil may also have a polar lipid content of about 45% by weight or about 50% by weight or even more.

WO 2010/104444 describes several methods of preparing oat kernel oil with a high polar lipid content by fractionation. The disclosure of WO 2010/104444 with respect to the preparation of oat kernel oil with a high polar lipid content is herewith incorporated by reference.

The cosmetic ingredient of the invention may be used to impart water resistance to cosmetic compositions, thereby protecting the cosmetic active(s) contained in the cosmetic compositions.

Therefore, the present invention also provides a cosmetic composition comprising the cosmetic ingredient and a cosmetic active.

The cosmetic composition of the invention may completely or essentially consist of the cosmetic ingredient and one or more cosmetic actives. Alternatively, the cosmetic composition may optionally further contain solvents, excipients, and/or other adjuvants. Any solvents, excipients, and/or other adjuvants commonly used in the preparation of cosmetic may be employed in the present invention.

In an embodiment, the cosmetic composition further comprises a cosmetically acceptable excipient.

Cosmetically acceptable excipients are well known in the art. Depending on the intended application, a suitable excipient may be selected.

Cosmetic compositions of the invention may contain one or more cosmetically acceptable excipients. Any excipients commonly used in the preparation of cosmetic preparations for use on the human skin or hair may be employed in the present invention. Suitable excipients include, but are not limited to, ingredients that can influence organoleptic properties, penetration of the skin, and the bioavailability of the cosmetic active agent of the present invention. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, and the like.

In an embodiment, the cosmetically acceptable excipient is selected from the group consisting of an oil-in-water emulsion, a water-in-oil emulsion, a lotion, a cream, an oil, a biphasic base, and an alcoholic base. These excipients have been found to be particularly well suited for use in sun protection skin or hair care compositions, for instance.

The cosmetic composition may take any physical form. For instance, the cosmetic composition may be in the form of a liposome composition, mixed liposomes, oleosomes, niosomes, ethosomes, milliparticles, microparticles, nanoparticles and solid-lipid nanoparticles, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the cosmetic active.

The cosmetic composition may be produced in any solid, liquid, or semi-solid form useful for topical application to the skin or hair or by transdermal application. Thus, these preparations for topical or transdermal application include, but are not restricted to, creams, multiple emulsions, such as and not restricted to, oil and/or silicone in water emulsions, water-in-oil and/or silicone emulsions, water/oil/water or water/silicone/water type emulsions, and oil/water/oil or silicone/water/silicone type emulsions, micro-emulsions, emulsions and/or solutions, liquid crystals, anhydrous compositions, oils, milks, balsams, foams, aqueous or oily lotions, oily gels, cream, hydro-alcoholic solutions, hydro-glycolic solutions, liniments, sera, soaps, face masks, serums, polysaccharide films, ointments, mousses, pomades, pastes, powders, bars, pencils and sprays or aerosols (sprays), including leave-on and rinse-off formulations.

Throughout this application, the terms “skin” and “hair” refer in particular to human skin and human hair, respectively.

The cosmetic ingredient should be included in the cosmetic composition in amount that is sufficient to provide the desired water protection effect. It has been found that concentrations of about 1% to about 5%, more preferably of about 2% to about 4%, and most preferably of about 3%, are particularly well suited.

Therefore in an embodiment, the cosmetic composition comprises the cosmetic ingredient in an amount of about 1% to about 5%, more preferably in an amount of about 2% to about 4%, and most preferably in an amount of about 3%.

A good water resistance is particularly important for cosmetic compositions that may be applied prior to contact with water, e.g. with sweat or sea water.

In an embodiment, the cosmetic composition is a skin or hair care composition, in particular a sun protection skin or hair care composition.

Sun protection compositions are particularly prone to activity loss caused by water, as people tend to sweat and/or use water to cool down, e.g. by bathing in the sea or a swimming pool. The cosmetic ingredient of the invention is able to improve their water resistance, thereby prolonging the sun protection.

In an embodiment, the cosmetic active is selected from the group consisting of a UV filter; an anti-aging active, such as e.g. sodium acetylated hyaluronate or retinol; an antioxidant, such as e.g. ferulic acid or vitamin E; a moisturizing active, such as e.g. sodium hyaluronate, porphyridium cruentum extract or hydrolyzed beta-glucan; a soothing active, such as e.g. bisabolol or Boswellia serrata gum; and mixtures thereof.

The cosmetic composition of the invention may comprise any suitable UV filter for protection of skin and/or hair. The UV filter may be physical or chemical or a combination thereof.

In an embodiment, the cosmetic composition comprises a UV filter selected from the group consisting of bis-ethylhexyloxyphenol methoxyphenyl triazine, butyl methoxydibenzoylmethane, diethylamino hydroxybenzoyl hexyl benzoate, disodium phenyl dibenzimidazole tetrasulfonate, drometrizole trisiloxane, menthyl anthranilate, methylene bis-benzotriazolyl tetramethylbutylphenol, terephthalylidene dicamphor sulfonic acid, zinc oxide, 4-methylbenzylidene camphor, benzophenone-3, benzophenone-4, diethylhexyl butamido triazone, ethylhexyl methoxycinnamate, ethylhexyl salicylate, ethylhexyl triazone, ethylhexyl dimethyl para-aminobenzoic acid, homomenthyl salicylate, isoamyl p-methoxycinnamate, octocrylene, polyoxyethylen (25) para-aminobenzoic acid, phenylbenzimidazol sulfonic acid, Polysilicone-15, titanium dioxide, tris biphenyl triazine, and mixtures thereof, more particularly a sun filter selected from the group consisting of bis-ethylhexyloxyphenol methoxyphenyl triazine, butyl methoxydibenzoylmethane, zinc oxide, ethylhexyl methoxycinnamate, ethylhexyl salicylate, ethylhexyl dimethyl para-aminobenzoic acid, homomenthyl salicylate, octocrylene, titanium dioxide, and mixtures thereof.

The above UV filters are generally known.

Bis-ethylhexyloxyphenol methoxyphenyl triazine is often abbreviated as BEMT.

Para-aminobenzoic acid is often abbreviated as PABA.

Ethylhexyl methoxycinnamate is often abbreviated as EHMC.

Ethylhexyl salicylate is often abbreviated as EHS.

Ethylhexyl triazone is often abbreviated as EHT.

Homomenthyl salicylate is often abbreviated as HMS.

Octocrylene is often abbreviated as OCR.

Polyoxyethylen(25) para-aminobenzoic acid is often abbreviated as PEG-25 PABA.

Polysilicone-15 (INCI) is an organic compound used in hair products like shampoos, conditioners, hair sprays, pomades and color treatment products to absorb UVB radiation. In the EU, it is also approved for use in sunscreens and cosmetics. The backbone chain is mainly composed of siloxane repeat units, and is of low molecular weight, so being classed as an oligomer. Its IUPAC name is α-(trimethylsilyl)-ω-(trimethylsilyloxy)poly[oxy(dimethyl)silylene]-co-[oxy(methyl)(2-{4-[2,2-bis(ethoxycarbonyl)vinyl]phenoxy}-1-methyleneethyl)silylene]-co-[oxy(methyl)(2-(4-[2,2-bis(ethoxycarbonyl)vinyl]phenoxy)prop-1-enyl)silylene].

The cosmetic composition of the invention may further comprise one or more adjuvants, such as a preservative, e.g. phenoxyethanol and/or ethylhexylglycerin; a humectant, e.g. glycerin and/or propylene glycol; a gelling agent, e.g. xanthan gum and/or cellulose gum; an emulsifier, e.g. sodium stearoyl glutamate and/or glyceryl stearate SE (SE=self emulsifying); a colorant, e.g. Cl 42090 (Blue 1) and/or CI 19140 (Yellow 5); and/or a fragrance.

The invention further relates to a method of improving the water resistance of a cosmetic composition, in particular of a sun protection skin or hair care composition, comprising the step of incorporating the cosmetic ingredient of the invention in the cosmetic composition.

The cosmetic ingredient may be simply added to and mixed with the cosmetic composition, or there may also be additional incorporation steps, such as heating or cooling, shaking or stirring, for instance. Preferably, the cosmetic ingredient is added to an oily phase, e.g. with gentle stirring, optionally at elevated temperature (e.g. 65-70° C.). The oily phase may then later be combined with one or more other phases, e.g. with an aqueous phase and/or an alcoholic phase.

The cosmetic ingredient may also be used as a film forming agent.

The cosmetic ingredient may further be used to improve the water resistance of a cosmetic composition.

The present invention is further illustrated by means of the following non-limiting examples:

### EXAMPLE 1: PROTOCOL FOR SPF MEASUREMENTS

The sun protection factor (SPF) was measured according to the following protocol:

### 1. 1. Weighing and Spreading

1.3 mg/cm² of the product to be tested was weighed using a precision 10⁻⁴ gram laboratory balance (AT200; Mettler Toledo). Then, the quantity was spread on the rough side of sandblasted Polymethyl metacrylate (PMMA) plates (Sunplates; Helioscience) according to the protocol described in the ISO 24443 method.

### 2. 2. Transmission Measurement

The transmission of PMMA plates was measured with a UV spectrophotometer (Uvikon 933; Bio-Tek Kontron Instruments, or UV-2600i; Shimadzu) equipped with a UV source and a monochromator capable of delivering UV energy between 290 and 400 nm.

### 3. 3. Irradiation

PMMA plates were exposed to UV radiation for 30 minutes, using a sun simulator (Suntest CPS+; Atlas) with an irradiance of 550 W/m². In this way, the tested product received two Minimal Erythemal Doses (MED).

### 4. 4. Transmission Measurement

The transmission of PMMA plates was measured again with a UV spectrophotometer (Uvikon 933; Bio-Tek Kontron Instruments, or UV-2600i; Shimadzu) equipped with a UV source and a monochromator capable of delivering UV energy between 290 and 400 nm.

### 5. 5. SPF Calculation

The SPF in vitro was expressed from the entire residual UVB and UVA spectrum that had passed through the layer of product spread on the PMMA plate. However, this wave function T (2) must be multiplied by:
A first wave function, which expresses the spectral characteristic of the sun S (2).
A second wave function, which expresses the reactivity of the skin as a function of the wavelength (and therefore of the dissipated energy): this is the erythematous function E (2).

$$SPF in vitro = \frac{{\sum }_{290 nm}^{400 nm} E ( λ ) , S ( λ ) , d λ}{{\sum }_{290 nm}^{400 nm} E ( λ ) , S ( λ ) , T ( λ ) , d λ}$$

Each product was measured three to five times. The mean SPF of the studied product was obtained by calculating the arithmetic mean of the protection indices of each test. Calculations were performed using specific software.

### EXAMPLE 2: ETHANOL OIL BASED SPRAY WITH SPF 20 (SYNTHETIC UV FILTERS)

The ability of different film formers to improve the water resistance was tested in an ethanol oil based spray formulation with a sun protection factor (SPF) of 20. They were tested in the following formulation containing synthetic UV filters:

| | | Trade | | Concentration |
|---|---|---|---|---|
| Phase | INCI name | name | Supplier | [wt %] |
| A | Diisopropyl Adipate, Propylene Glycol | Dub | Stearinerie | SQ 100 |
| | Dicaprylate/Dicaprate, C12-15 Alkyl | Helioptima | Dubois | |
| | Benzoate, Diisopropyl Sebacate | | | |
| | Bis-Ethylhexyloxyphenol | Escalol S | Ashland | 6.2 |
| | Methoxyphenyl Triazine | | | |
| | Butyl Methoxybenzoylmethane | Escalol 517 | Ashland | 3.0 |
| | Ethylhexyl Salicylate | Escalol 587 | Ashland | 5.0 |
| B | Tocopheryl Acetate | Vitamin E | BASF | 0.3 |
| | | acetate | | |
| | [Film former] | | | 0-5 |
| C | Alcohol | Alcohol | Dislaub | 19.9 |
| | | 99% | | |

The spray formulations to be tested were prepared by heating phase A to 80° C. for one hour under gentle stirring, cooling it down to 70° C. and adding phase B under gentle stirring, and cooling it down to 25° C. and adding phase C under gentle stirring.

At the outset, the SPF was measured for each spray formulation as described in example 1.

The PMMA plates with the spray formulations were then immersed in a water bath of thermostatically controlled running water (30±2° C.) equipped with an adjustable flow water circulation pump (Polystat 86694; Bioblock Scientific) for 20 min. After the bath, the SPF was measured again.

The water resistance was determined according to the following formula:

$$Water resistance ( \% ) = \frac{SPF after bath}{SPF before bath} * 100$$

The following film formers were tested alone or in combination:

| Trade name | INCI name | Supplier | Origin |
|---|---|---|---|
| Dermacryl 79 | Acrylates/Octylacrylamide Copolymer | Nouryon | Synthetic |
| Unimer U-151 | VP/Hexadecene Copolymer | Givaudan | Synthetic |
| SolAmaze Sun Natural MB | Diisostearoyl Polyglyceryl-3 Dimer | Nouryon | Natural |
| | Dilinoleate, Caprylic/Capric Triglyceride | | |
| Lexfilm Sun Natural MB | Capryloyl Glycerin/Sebacic Acid | Inolex | Natural |
| | Copolymer | | |
| Sweoat ® Oil PL40 FG | Avena Sativa (Oat) Kernel Oil (with polar | Oat | Natural |
| | lipids content ≥ 40%) | Cosmetics | |
| Boswellia Thick Oil | Boswellia Serrata Oil | Indfrag | Natural |
| | | Biosciences | |
| Emolid DD | Dimer Dilinoleyl Dimer Dilinoleate | IQL | Natural |
| Emolid CC* | Coco-Caprylate/Caprate | IQL | Natural |

*SPF enhancer

The results are shown in Table 1 below (film formers in brackets are mixtures, and the numbers afterwards indicate the weight ratio).

These results show that:
5% of Unimer U-151 is better than 3% of Unimer U-151
Dermacryl 79 is more efficient than Unimer U-151
Sweoat® Oil PL40 FG is better than Boswellia Thick Oil
SolAmaze Sun Natural MB, Lexfilm Sun Natural MB, Emolid DD, Emolid CC, and mixtures of Boswellia Thick Oil or Sweoat® Oil PL40 FG with Emolid DD and Emolid CC do not improve the water resistance
There is a good synergy between Sweoat® Oil PL40 FG and Boswellia Thick Oil
The best results are observed for a 40:60 mixture of Boswellia Thick Oil and Sweoat® Oil PL40 FG
A concentration of about 3% provides the best results
Boswellia thick oil prepared by extraction with ethanol provides similar results as Boswellia thick oil prepared by hexane extraction

### EXAMPLE 3: ETHANOL OIL BASED SPRAY WITH SPF 30 (SYNTHETIC UV FILTERS)

The ability of a 40:60 mixture of Boswellia Thick Oil and oat kernel oil to improve the water resistance was tested in an ethanol oil based spray formulation with a sun protection factor (SPF) of 30. They were tested in the following formulation containing synthetic UV filters:

| | | | | Concentration |
|---|---|---|---|---|
| Phase | INCI name | Trade name | Supplier | [wt %] |
| A | Diisopropyl Adipate, Propylene | Dub | Stearinerie | SQ 100 |
| | Glycol Dicaprylate/Dicaprate, | Helioptima | Dubois | |
| | C12-15 Alkyl Benzoate, | | | |
| | Diisopropyl Sebacate | | | |
| | Butyl Methoxybenzoylmethane | Escalol 517 | Ashland | 3.0 |
| | Homosalate | Escalol HMS | Ashland | 10 |
| | Octocrylene | Escalol 597 | Ashland | 10 |
| | Ethylhexyl Dimethyl PABA | Escalol 507 | Ashland | 0.9 |
| | Ethylhexyl Salicylate | Escalol 587 | Ashland | 5.0 |
| B | Tocopheryl Acetate | Vitamin E | BASF | 0.3 |
| | | acetate | | |
| | Boswellia serrata oil, Avena | Boswellia | Indfrag | 0-5 |
| | Sativa (Oat) Kernel Oil | thick Oil + | Biosciences / | |
| | | Sweoat ® Oil | Oat | |
| | | PL40 FG | Cosmetics | |
| | | (40/60) | | |
| C | Alcohol | Alcohol 99% | Dislaub | 19.9 |

Testing was done as described in example 2 above.

The results are shown in Table 2 below.

As can be seen from those results, a 40:60 mixture of Boswellia Thick Oil and Sweoat® Oil PL40 FG at a concentration of about 3% provides the best results.

### EXAMPLE 4: ETHANOL OIL BASED SPRAY WITH SPF 50 (SYNTHETIC UV FILTERS)

The ability of a 40:60 mixture of Boswellia Thick Oil and oat kernel oil to improve the water resistance was tested in an ethanol oil based spray formulation with a sun protection factor (SPF) of 50. They were tested in the following formulation containing synthetic UV filters:

| | | | | Concentration |
|---|---|---|---|---|
| Phase | INCI name | Trade name | Supplier | [wt %] |
| A | Diisopropyl Adipate, Propylene | Dub | Stearinerie | SQ 100 |
| | Glycol Dicaprylate/Dicaprate, | Helioptima | Dubois | |
| | C12-15 Alkyl Benzoate, | | | |
| | Diisopropyl Sebacate | | | |
| | Butyl Methoxybenzoylmethane | Escalol 517 | Ashland | 3.0 |
| | Homosalate | Escalol HMS | Ashland | 10 |
| | Octocrylene | Escalol 597 | Ashland | 10 |
| | Ethylhexyl Dimethyl PABA | Escalol 507 | Ashland | 6.0 |
| | Ethylhexyl Salicylate | Escalol 587 | Ashland | 5.0 |
| | Ethylhexyl Methoxycinnamate | Uvinul MC 80 | Ashland | 1.0 |
| | Bis-Ethylhexyloxyphenol | Escalol S | Ashland | 7.0 |
| | Methoxyphenyl Triazine | | | |
| B | Tocopheryl Acetate | Vitamin E | BASF | 0.3 |
| | | acetate | | |
| | Boswellia serrata oil, Avena | Boswellia | Indfrag | 0-5 |
| | Sativa (Oat) Kernel Oil | thick Oil + | Biosciences / | |
| | | Sweoat ® Oil | Oat | |
| | | PL40 FG | Cosmetics | |
| | | (40/60) | | |
| C | Alcohol | Alcohol 99% | Dislaub | 19.9 |

Testing was done as described in example 2 above.

The results are shown in Table 3 below.

As can be seen from those results, a 40:60 mixture of Boswellia Thick Oil and Sweoat® Oil PL40 FG at a concentration of about 3% provides the best results.

### EXAMPLE 5: SUNSCREEN WITH SPF 20 (SYNTHETIC UV FILTERS)

The ability of different film formers to improve the water resistance was tested in a sunscreen formulation with a sun protection factor (SPF) of 20. They were tested in the following formulation containing synthetic UV filters:

| | | | | Concentration |
|---|---|---|---|---|
| Phase | INCI name | Trade name | Supplier | [wt %] |
| A | Diisopropyl Adipate, Propylene | Dub | Stearinerie | 16 |
| | Glycol Dicaprylate/Dicaprate, C12- | Helioptima | Dubois | |
| | 15 Alkyl Benzoate, Diisopropyl | | | |
| | Sebacate | | | |
| | Bis-Ethylhexyloxyphenol | Escalol S | Ashland | 6.2 |
| | Methoxyphenyl Triazine | | | |
| | Butyl Methoxybenzoylmethane | Escalol 517 | Ashland | 3.0 |
| | Ethylhexyl Salicylate | Escalol 587 | Ashland | 5.0 |
| B | Sucrose Stearate | Sisterna SP | Sisterna BV | 1.0 |
| | | 70 C | | |
| | Tocopherol | Covi ox | BASF | 0.1 |
| | | T70C | | |
| C | [Film former] | | | 0-5 |
| D | Water | | | SQ 100 |
| | Microcrystalline Cellulose, Cellulose | Avicel PC | FMC | 1.2 |
| | Gum | 611 | Corporation | |
| E | Glycerin | Vegetal | Oleon | 1.5 |
| | | glycerin | | |
| | | rapeseed | | |
| | Xanthan Gum | Keltrol CG- | CP Kelco | 0.3 |
| | | SFT | | |
| F | Sodium Phytate, Water, Alcohol | Dermofeel | Evonik | 0.1 |
| | | PA-3 | | |
| | Glyceryl Stearate Citrate | Dermosoft | Evonik | 3.0 |
| | | GSC POF | | |
| G | Pentylene Glycol | A-Leen 5 | Minasolve | 5.0 |
| H | Citric Acid | Citric Acid | Brenntag | QS pH 5.5 |

The sunscreen formulations to be tested were prepared by preparing and heating phase A to 80° C. for 30 minutes, preparing phase D under high stirring at 5000 rpm for 30 min, adding the premixed phase E to D under gentle stirring and heating it to 80° C., adding phases B and C to phase A and then phase F to the mixture of phases D&E under gentle stirring just before emulsification, adding the mixture of phases D&E&F to the mixture of phases A&B&C under high stirring at 3000 rpm for a few minutes, cooling down to 30° C. under gentle stirring, adding phase G, and adjusting the pH to 5.5 with phase H.

Testing was done as described in example 2 above.

The results are shown in Table 4 below.

These results show that:
Only mixtures of Boswellia thick oil and oat kernel oil led to a better water resistance compared to the formulation without film former
The best results are observed for 30:70, 35:65, and 40:60 mixtures of Boswellia Thick Oil and Sweoat® Oil PL40 FG
Mixtures of Boswellia Thick Oil and Sweoat® Oil PL40 FG with a ratio of between 25:75 to 45:55 provide a water resistance of 100% or higher
A concentration of about 3% provides the best results
Boswellia thick oil prepared by extraction with ethanol provides similar results as Boswellia thick oil prepared by hexane extraction

### EXAMPLE 6: SUNSCREEN WITH SPF 30 (SYNTHETIC UV FILTERS)

The ability of a 40:60 mixture of Boswellia Thick Oil and oat kernel oil to improve the water resistance was tested in a sunscreen formulation with a sun protection factor (SPF) of 30. They were tested in the following formulation containing synthetic UV filters:

| | | | | Concentration |
|---|---|---|---|---|
| Phase | INCI name | Trade name | Supplier | [wt %] |
| A | Diisopropyl Adipate, Propylene | Dub Helioptima | Stearinerie | 7.0 |
| | Glycol Dicaprylate/Dicaprate, | | Dubois | |
| | C12-15 Alkyl Benzoate, | | | |
| | Diisopropyl Sebacate | | | |
| | Butyl Methoxybenzoylmethane | Escalol 517 | Ashland | 3.0 |
| | Homosalate | Escalol HMS | Ashland | 10 |
| | Octocrylene | Escalol 597 | Ashland | 10 |
| | Ethylhexyl Dimethyl PABA | Escalol 507 | Ashland | 0.9 |
| | Ethylhexyl Salicylate | Escalol 587 | Ashland | 5.0 |
| B | Sucrose Stearate | Sisterna SP 70 C | Sisterna BV | 1.0 |
| | Tocopherol | Covi ox T70C | BASF | 0.1 |
| C | Boswellia serrata oil, Avena | Boswellia thick | Indfrag | 0-3 |
| | Sativa (Oat) Kernel Oil | Oil + Sweoat ® Oil | Biosciences/ | |
| | | PL40 FG (40/60) | Oat | |
| | | | Cosmetics | |
| D | Water | | | QS 100 |
| | Microcrystalline Cellulose, | Avicel PC 611 | FMC | 1.2 |
| | Cellulose Gum | | Corporation | |
| E | Glycerin | Vegetal glycerin | Oleon | 1.5 |
| | | rapeseed | | |
| | Xanthan Gum | Keltrol CG-SFT | CP Kelco | 0.3 |
| F | Sodium Phytate, Water, Alcohol | Dermofeel PA-3 | Evonik | 0.1 |
| | Glyceryl Stearate Citrate | Dermosoft GSC | Evonik | 3.0 |
| | | POF | | |
| G | Pentylene Glycol | A-Leen 5 | Minasolve | 5.0 |
| H | Citric Acid | Citric Acid | Brenntag | QS pH 5.5 |

Testing was done as described in example 5 above.

The results are shown in Table 5 below.

As can be seen from those results, a 40:60 mixture of Boswellia Thick Oil and Sweoat® Oil PL40 FG at a concentration of about 3% is clearly better than the control without a film former.

### EXAMPLE 7: SUNSCREEN WITH SPF 50 (SYNTHETIC UV FILTERS)

The ability of a 40:60 mixture of Boswellia Thick Oil and oat kernel oil to improve the water resistance was tested in a sunscreen formulation with a sun protection factor (SPF) of 50. They were tested in the following formulation containing synthetic UV filters:

| | | | | Concentration |
|---|---|---|---|---|
| Phase | INCI name | Trade name | Supplier | [wt %] |
| A | Diisopropyl Adipate, Propylene | Dub | Stearinerie | 4.0 |
| | Glycol Dicaprylate/Dicaprate, | Helioptima | Dubois | |
| | C12-15 Alkyl Benzoate, | | | |
| | Diisopropyl Sebacate | | | |
| | Butyl Methoxybenzoylmethane | Escalol 517 | Ashland | 3.0 |
| | Homosalate | Escalol HMS | Ashland | 10 |
| | Octocrylene | Escalol 597 | Ashland | 10 |
| | Ethylhexyl Dimethyl PABA | Escalol 507 | Ashland | 6.0 |
| | Ethylhexyl Salicylate | Escalol 587 | Ashland | 5.0 |
| | Ethylhexyl Methoxycinnamate | Uvinul MC 80 | Ashland | 1.0 |
| | Bis-Ethylhexyloxyphenol | Escalol S | Ashland | 7.0 |
| | Methoxyphenyl Triazine | | | |
| B | Sucrose Stearate | Sisterna SP | Sisterna BV | 1.0 |
| | | 70 C | | |
| | Tocopherol | Covi ox T70C | BASF | 0.1 |
| C | Boswellia serrata oil, Avena | Boswellia | Indfrag | 0-3 |
| | Sativa (Oat) Kernel Oil | thick Oil | Biosciences / | |
| | | Sweoat ® Oil | Oat | |
| | | PL40 FG | Cosmetics | |
| | | (40/60) | | |
| D | Water | | | QS 100 |
| | Microcrystalline Cellulose, | Avicel PC 611 | FMC | 1.2 |
| | Cellulose Gum | | Corporation | |
| E | Glycerin | Vegetal | Oleon | 1.5 |
| | | glycerin | | |
| | | rapeseed | | |
| | Xanthan Gum | Keltrol CG- | CP Kelco | 0.3 |
| | | SFT | | |
| F | Sodium Phytate, Water, Alcohol | Dermofeel | Evonik | 0.1 |
| | | PA-3 | | |
| | Glyceryl Stearate Citrate | Dermosoft | Evonik | 3.0 |
| | | GSC POF | | |
| G | Pentylene Glycol | A-Leen 5 | Minasolve | 5.0 |
| H | Citric Acid | Citric Acid | Brenntag | QS pH 5.5 |

Testing was done as described in example 5 above.

The results are shown in Table 6 below.

As can be seen from those results, a 40:60 mixture of Boswellia Thick Oil and Sweoat® Oil PL40 FG at a concentration of about 3% is clearly better than the control without a film former.

### EXAMPLE 8: SUNSCREEN WITH SPF 20 (MINERAL UV FILTERS)

The ability of different film formers to improve the water resistance was tested in a sunscreen formulation with a sun protection factor (SPF) of 20. They were tested in the following formulation containing mineral UV filters:

| | | | | Concentration |
|---|---|---|---|---|
| Phase | INCI name | Trade name | Supplier | [wt %] |
| A | Diisopropyl Adipate, Propylene | Dub | Stearinerie | 8.0 |
| | Glycol Dicaprylate/Dicaprate, C12- | Helioptima | Dubois | |
| | 15 Alkyl Benzoate, Diisopropyl | | | |
| | Sebacate | | | |
| A | Titanium Dioxide, Caprylic/Capric | UV CUT | Grant | 12.8 |
| | Triglyceride, Stearic Acid, Alumina, | TiO2-55-CG | industries | |
| | Polyhydroxystearic Acid | | | |
| | Zinc Oxide, Caprylic/Capric | UV CUT | Grant | 14.8 |
| | Triglyceride, Polyhydroxystearic | ZnO-68-CG | industries | |
| | Acid | | | |
| B | Sucrose Stearate | Sisterna SP | Sisterna | 1.0 |
| | | 70 C | BV | |
| | Tocopherol | Covi ox | BASF | 0.1 |
| | | T70C | | |
| C | [Film former] | | | 0-3 |
| D | Water | | | SQ 100 |
| | Microcrystalline Cellulose, Cellulose | Avicel PC | FMC | 1.2 |
| | Gum | 611 | Corporation | |
| E | Glycerin | Vegetal | Oleon | 1.5 |
| | | glycerin | | |
| | | rapeseed | | |
| | Xanthan Gum | Keltrol CG- | CP Kelco | 0.3 |
| | | SFT | | |
| F | Sodium Phytate, Water, Alcohol | Dermofeel | Evonik | 0.1 |
| | | PA-3 | | |
| | Glyceryl Stearate Citrate | Dermosoft | Evonik | 3.0 |
| | | GSC POF | | |
| G | Pentylene Glycol | A-Leen 5 | Minasolve | 5.0 |
| H | Citric Acid | Citric Acid | Brenntag | QS pH 5.5 |

The sunscreen formulations to be tested were prepared by preparing and heating phase A to 80° C. and then mixing it at 3000 rpm for 10 min, preparing phase D under high stirring at 5000 rpm for 30 min, adding the premixed phase E to D under gentle stirring and heating it to 80° C., adding phases B and C to phase A and then phase F to the mixture of phases D&E under gentle stirring just before emulsification, adding the mixture of phases D&E&F to the mixture of phases A&B&C under high stirring at 3000 rpm for a few minutes, cooling down to 30° C. under gentle stirring, adding phase G, and adjusting the pH to 5.5 with phase H.

Testing was done as described in example 2 above.

The results are shown in Table 7 below.

As can be seen from those results, both Unimer U-151 and a 40:60 mixture of Boswellia Thick Oil and Sweoat® Oil PL40 FG at a concentration of about 3% each are better than the control without a film former.

### EXAMPLE 9: SUNSCREEN WITH SPF 30 (SYNTHETIC AND MINERAL UV FILTERS)

The ability of a 40:60 mixture of Boswellia Thick Oil and oat kernel oil to improve the water resistance was tested in a sunscreen formulation with a sun protection factor (SPF) of 30. They were tested in the following formulation containing synthetic and mineral UV filters:

| | | | | Concentration |
|---|---|---|---|---|
| Phase | INCI name | Trade name | Supplier | [wt %] |
| A | Diisopropyl Adipate, Propylene | Dub | Stearinerie | 3.0 |
| | Glycol Dicaprylate/Dicaprate, | Helioptima | Dubois | |
| | C12-15 Alkyl Benzoate, | | | |
| | Diisopropyl Sebacate | | | |
| | Butyl Methoxybenzoylmethane | Escalol 517 | Ashland | 1.0 |
| | Homosalate | Escalol HMS | Ashland | 5.0 |
| | Ethylhexyl Dimethyl PABA | Escalol 507 | Ashland | 3.0 |
| | Ethylhexyl Salicylate | Escalol 587 | Ashland | 5.0 |
| B | Titanium Dioxide, Caprylic/Capric | UV CUT TIO2- | Grant | 9.1 |
| | Triglyceride, Stearic Acid, | 55-CG | industries | |
| | Alumina, Polyhydroxystearic Acid | | | |
| | Zinc Oxide, Caprylic/Capric | UV CUT ZnO- | Grant | 7.4 |
| | Triglyceride, Polyhydroxystearic | 68-CG | industries | |
| | Acid | | | |
| C | Sucrose Stearate | Sisterna SP | Sisterna BV | 1.0 |
| | | 70 C | | |
| | Tocopherol | Covi ox T70C | BASF | 0.1 |
| D | Avena Sativa (Oat) Kernel Oil, | Boswellia | Indfrag | 0-3 |
| | Boswellia serrata oil | thick Oil + | Biosciences / | |
| | | Sweoat ® Oil | Oat | |
| | | PL40 FG | Cosmetics | |
| | | (40/60) | | |
| E | Water | | | QS 100 |
| | Microcrystalline Cellulose, | Avicel PC 611 | FMC | 1.2 |
| | Cellulose Gum | | Corporation | |
| F | Glycerin | Vegetal | Oleon | 1.5 |
| | | glycerin | | |
| | | rapeseed | | |
| | Xanthan Gum | Keltrol CG- | CP Kelco | 0.3 |
| | | SFT | | |
| G | Sodium Phytate, Water, Alcohol | Dermofeel | Evonik | 0.1 |
| | | PA-3 | | |
| | Glyceryl Stearate Citrate | Dermosoft | Evonik | 3.0 |
| | | GSC POF | | |
| H | Pentylene Glycol | A-Leen 5 | Minasolve | 5.0 |
| - | Citric Acid | Citric Acid | Brenntag | QS pH 5.5 |

The sunscreen formulations to be tested were prepared by preparing and heating phase A to 80° C. for 30 minutes, adding phase B to phase A at 80° C. under gentle stirring and then mixing it at 3000 rpm for 10 min, preparing phase E under high stirring at 5000 rpm for 30 min, adding the premixed phase F to E under gentle stirring and heating it to 80° C., adding phases C and D to the mixture of phases A&B and then phase G to the mixture of phases E&F under gentle stirring just before emulsification, adding the mixture of phases E&F&G to the mixture of phases A&B&C&D under high stirring at 3000 rpm for a few minutes, cooling down to 30° C. under gentle stirring, adding phase H, and adjusting the pH to 5.5 with phase I.

Testing was done as described in example 2 above.

The results are shown in Table 8 below.

As can be seen from those results, a 40:60 mixture of Boswellia Thick Oil and Sweoat® Oil PL40 FG at a concentration of about 3% is clearly better than the control without a film former.

TABLE 1
Film formers in ethanol oil based spray with SPF 20 (synthetic UV filters)

| | SPF before bath | | SPF after bath | | | Average |
|---|---|---|---|---|---|---|
| | | Standard | | Standard | Water | water |
| Film former | Mean | deviation | Mean | deviation | resistance | resistance |
| Without film former | 22.0 | 2.1 | 18.5 | 1.8 | 84% | 86% |
| | 26.5 | 0.9 | 21.9 | 1.3 | 83% | |
| | 23.8 | 0.1 | 22.0 | 1.3 | 92% | |
| 1% Dermacryl 79 | 22.8 | 1.5 | 24.8 | 0.2 | 109% | 109% |
| 3% Dermacryl 79 | 23.5 | 2.3 | 24.0 | 1.7 | 102% | 101% |
| | 28.2 | 2.1 | 30.0 | 2.8 | 106% | |
| | 25.7 | 1.1 | 24.5 | 0.1 | 95% | |
| 5% Dermacryl 79 | 25.1 | 0.1 | 26.2 | 1.0 | 104% | 104% |
| 3% Unimer U-151 | 20.2 | 4.2 | 17.3 | 1.9 | 86% | 86% |
| 5% Unimer U-151 | 21.3 | 3.5 | 19.4 | 1.8 | 91% | 94% |
| | 24.3 | 0.7 | 23.5 | 0.4 | 97% | |
| 3% SolAmaze | 20.0 | 2.1 | 16.8 | 0.6 | 84% | 84% |
| Sun Natural MB | | | | | | |
| 3% Lexfilm | 23.8 | 0.7 | 21.1 | 0.3 | 89% | 89% |
| Sun Natural MB | | | | | | |
| 3% Sweoat ® | 29.2 | 2.6 | 24.9 | 1.9 | 85% | 85% |
| Oil PL40 FG | | | | | | |
| 3% Boswellia | 26.2 | 4.2 | 20.4 | 1.9 | 78% | 78% |
| Thick Oil | | | | | | |
| 3% Emolid DD | 29.1 | 7.4 | 25.8 | 3.8 | 88% | 88% |
| 3% Emolid CC | 24.2 | 2.1 | 20.3 | 0.7 | 84% | 84% |
| 3% (Boswellia | 26.8 | 2.6 | 27.3 | 0.3 | 102% | 104% |
| Thick Oil + | 20.0 | 2.9 | 21.1 | 2.4 | 105% | |
| Sweoat ® Oil | | | | | | |
| PL40 FG) | | | | | | |
| (50:50) | | | | | | |
| 3% (Boswellia | 27.2 | 3.1 | 26.4 | 2.6 | 97% | 97% |
| Thick Oil + | | | | | | |
| Sweoat ® Oil | | | | | | |
| PL40 FG + | | | | | | |
| Emolid DD) | | | | | | |
| (40:40:20) | | | | | | |
| 3% (Boswellia | 22.4 | 4.3 | 21.4 | 3.3 | 96% | 96% |
| Thick Oil + | | | | | | |
| Sweoat ® Oil | | | | | | |
| PL40 FG + | | | | | | |
| Emolid DD + | | | | | | |
| Emolid CC) | | | | | | |
| (25:25:25:25) | | | | | | |
| 3% (Boswellia | 24.9 | 3.3 | 18.9 | 0.7 | 76% | 76% |
| Thick Oil + | | | | | | |
| Emolid DD + | | | | | | |
| Emolid CC) | | | | | | |
| (50:25:25) | | | | | | |
| 3% Sweoat ® | 29.9 | 8.2 | 23.6 | 3.2 | 79% | 79% |
| Oil PL40 FG + | | | | | | |
| Emolid DD + | | | | | | |
| Emolid CC) | | | | | | |
| (50:25:25) | | | | | | |
| 3% (Boswellia | 25.2 | 6.4 | 25.9 | 1.6 | 103% | 103% |
| Thick Oil + | | | | | | |
| Sweoat ® Oil | | | | | | |
| PL40 FG) | | | | | | |
| (45:55) | | | | | | |
| 3% (Boswellia | 22.2 | 4.4 | 24.8 | 5.1 | 112% | 108% |
| Thick Oil + | 20.8 | 0.8 | 22.2 | 2.5 | 107% | |
| Sweoat ® Oil | 24.8 | 1.6 | 25.2 | 2.3 | 102%* | |
| PL40 FG) | | | | | | |
| (40:60) | | | | | | |
| 3% (Boswellia | 24.0 | 1.0 | 27.1 | 3.0 | 113% | |
| Thick Oil + | | | | | | |
| Sweoat ® Oil | | | | | | |
| PL40 FG) | | | | | | |
| (40:60) after 1 | | | | | | |
| month at 20° C. | | | | | | |
| 3% (Boswellia | 23.1 | 1.0 | 24.3 | 1.0 | 105% | |
| Thick Oil + | | | | | | |
| Sweoat ® Oil | | | | | | |
| PL40 FG) | | | | | | |
| (40:60), | | | | | | |
| deodorized | | | | | | |
| 3% (Boswellia | 27.7 | 6.0 | 29.5 | 4.1 | 107% | 107% |
| Thick Oil + | | | | | | |
| Sweoat ® Oil | | | | | | |
| PL40 FG) | | | | | | |
| (25:75) | | | | | | |
| 1% (Boswellia | 23.5 | 3.8 | 20.9 | 2.0 | 89% | 93% |
| Thick Oil + | 26.3 | 4.3 | 24.4 | 1.6 | 93% | |
| Sweoat ® Oil | 24.9 | 2.4 | 24.1 | 0.9 | 97%* | |
| PL40 FG) | | | | | | |
| (40:60) | | | | | | |
| 2% (Boswellia | 26.6 | 7.2 | 22.7 | 4.7 | 85% | 92% |
| Thick Oil + | 20.1 | 0.2 | 19.7 | 2.6 | 98% | |
| Sweoat ® Oil | | | | | | |
| PL40 FG) | | | | | | |
| (40:60) | | | | | | |
| 4% (Boswellia | 22.0 | 0.5 | 18.3 | 2.7 | 83% | 82% |
| Thick Oil + | 24.4 | 1.6 | 19.7 | 2.6 | 81% | |
| Sweoat ® Oil | | | | | | |
| PL40 FG) | | | | | | |
| (40:60) | | | | | | |
| 5% (Boswellia | 29.5 | 3.8 | 25.9 | 2.7 | 88% | 93% |
| Thick Oil + | 28.1 | 0.8 | 25.0 | 3.9 | 89% | |
| Sweoat ® Oil | 21.7 | 1.6 | 22.1 | 0.4 | 102%* | |
| PL40 FG) | | | | | | |
| (40:60) | | | | | | |

*Boswellia Thick Oil obtained from ethanol extraction

TABLE 2
Film formers in ethanol oil based spray
with SPF 30 (synthetic UV filters)

| | SPF before bath | | SPF after bath | | |
|---|---|---|---|---|---|
| | | Standard | | Standard | Water |
| Film former | Mean | deviation | Mean | deviation | resistance |
| Without film | 35.4 | 0.5 | 24.4 | 0.3 | 69% |
| former | | | | | |
| 1% (Boswellia | 38.3 | 0.6 | 27.7 | 0.9 | 72% |
| Thick Oil + | | | | | |
| Sweoat ® Oil | | | | | |
| PL40 FG) | | | | | |
| (40:60) | | | | | |
| 3% (Boswellia | 39.3 | 2.3 | 46.0 | 1.1 | 117% |
| Thick Oil + | | | | | |
| Sweoat ® Oil | | | | | |
| PL40 FG) | | | | | |
| (40:60) | | | | | |
| 5% (Boswellia | 39.5 | 2.4 | 33.8 | 1.4 | 86% |
| Thick Oil + | | | | | |
| Sweoat ® Oil | | | | | |
| PL40 FG) | | | | | |
| (40:60) | | | | | |

TABLE 3
Film formers in ethanol oil based spray
with SPF 50 (synthetic UV filters)

| | SPF before bath | | SPF after bath | | |
|---|---|---|---|---|---|
| | | Standard | | Standard | Water |
| Film former | Mean | deviation | Mean | deviation | resistance |
| Without film former | 56.1 | 0.9 | 55.2 | 0.6 | 98% |
| 1% (Boswellia | 59.2 | 0.5 | 56.2 | 1.3 | 95% |
| Thick Oil + | | | | | |
| Sweoat ® Oil PL40 | | | | | |
| FG) (40:60) | | | | | |
| 3% (Boswellia | 50.3 | 2.9 | 61.4 | 6.2 | 122% |
| Thick Oil + | | | | | |
| Sweoat ® Oil PL40 | | | | | |
| FG) (40:60) | | | | | |
| 5% (Boswellia | 57.2 | 3.9 | 56.1 | 4.2 | 98% |
| Thick Oil + | | | | | |
| Sweoat ® Oil PL40 | | | | | |
| FG) (40:60) | | | | | |

TABLE 4
Film formers in sunscreen with SPF 20 (synthetic UV filters)

| | SPF before bath | | SPF after bath | | | Average |
|---|---|---|---|---|---|---|
| | | Standard | | Standard | Water | water |
| Film former | Mean | deviation | Mean | deviation | resistance | resistance |
| Without film former | 23.7 | 10.5 | 17.5 | 7.9 | 74% | 81% |
| | 19.0 | 0.8 | 16.4 | 3.5 | 87% | |
| 3% Unimer U-151 | 23.9 | 4.6 | 14.4 | 5.7 | 61% | 62% |
| | 24.1 | 4.4 | 14.9 | 3.3 | 62% | |
| 3% Symeffect Sun** | 24.3 | 3.0 | 22.7 | 2.5 | 93% | 93% |
| 3% Lexfilm Sun | 19.6 | 3.2 | 16.0 | 3.9 | 82% | 82% |
| Natural MB | | | | | | |
| 3% Emolid DD | 21.9 | 0.7 | 17.02 | 1.2 | 76% | 76% |
| 3% (Boswellia | 20.4 | 0.6 | 18.4 | 2.5 | 90% | 96% |
| Thick Oil + | 19.0 | 3.2 | 19.5 | 2.4 | 103% | |
| Sweoat ® Oil PL40 | | | | | | |
| FG) (50:50) | | | | | | |
| 3% (Boswellia | 19.3 | 1.5 | 14.1 | 1.6 | 73% | 73% |
| Thick Oil + | | | | | | |
| Sweoat ® Oil PL40 | | | | | | |
| FG + Emolid DD) | | | | | | |
| (40:40:20) | | | | | | |
| 3% (Boswellia | 17.4 | 3.8 | 17.4 | 5.4 | 100% | 100% |
| Thick Oil + | | | | | | |
| Sweoat ® Oil PL40 | | | | | | |
| FG) (45:55) | | | | | | |
| 3% (Boswellia | 19.5 | 2.1 | 21.8 | 4.0 | 112% | 109% |
| Thick Oil + | 18.9 | 0.9 | 19.4 | 1.9 | 103%* | |
| Sweoat ® Oil PL40 | | | | | | |
| FG) (40:60) | | | | | | |
| 3% (Boswellia | 19.0 | 1.1 | 21.2 | 0.6 | 111% | |
| Thick Oil + | | | | | | |
| Sweoat ® Oil PL40 | | | | | | |
| FG) (40:60) after 1 | | | | | | |
| month at 20° C. | | | | | | |
| 3% (Boswellia | 19.2 | 2.3 | 21.9 | 2.2 | 114% | 114% |
| Thick Oil + | | | | | | |
| Sweoat ® Oil PL40 | | | | | | |
| FG) (35:65) | | | | | | |
| 3% (Boswellia | 23.9 | 2.1 | 28.2 | 1.9 | 118% | 118% |
| Thick Oil + | | | | | | |
| Sweoat ® Oil PL40 | | | | | | |
| FG) (30:70) | | | | | | |
| 3% (Boswellia | 18.4 | 3.3 | 20.0 | 4.3 | 109% | 109% |
| Thick Oil + | | | | | | |
| Sweoat ® Oil PL40 | | | | | | |
| FG) (25:75) | | | | | | |
| 1% (Boswellia | 25.3 | 4.9 | 23.9 | 4.7 | 95% | 95% |
| Thick Oil + | | | | | | |
| Sweoat ® Oil PL40 | | | | | | |
| FG) (40:60) | | | | | | |
| 5% (Boswellia | 18.4 | 0.3 | 19.5 | 0.3 | 106% | 106% |
| Thick Oil + | | | | | | |
| Sweoat ® Oil PL40 | | | | | | |
| FG) (40:60) | | | | | | |

*Boswellia Thick Oil obtained from ethanol extraction
**Symeffect Sun: Beeswax (Cera Alba), Sodium Stearoyl Lactylate

TABLE 5
Film formers in sunscreen with SPF 30 (synthetic UV filters)

| | SPF before bath | | SPF after bath | | |
|---|---|---|---|---|---|
| | | Standard | | Standard | Water |
| Film former | Mean | deviation | Mean | deviation | resistance |
| Without film | 43.8 | 1.3 | 33.8 | 2.1 | 77% |
| former | | | | | |
| 3% | 37.4 | 3.3 | 33.1 | 3.4 | 88% |
| (Boswellia | | | | | |
| Thick Oil + | | | | | |
| Sweoat ® Oil | | | | | |
| PL40 FG) | | | | | |
| (40:60) | | | | | |

TABLE 6
Film formers in sunscreen with SPF 50 (synthetic UV filters)

| | SPF before bath | | SPF after bath | | |
|---|---|---|---|---|---|
| | | Standard | | Standard | Water |
| Film former | Mean | deviation | Mean | deviation | resistance |
| Without film | 53.1 | 0.5 | 44.1 | 0.9 | 83% |
| former | | | | | |
| 3% (Boswellia | 46.3 | 1.2 | 47.3 | 2.4 | 102% |
| Thick Oil | | | | | |
| Sweoat ® Oil | | | | | |
| PL40 FG) | | | | | |
| (40:60) | | | | | |

TABLE 7
Film formers in sunscreen with SPF 20 (mineral UV filters)

| | SPF before bath | | SPF after bath | | |
|---|---|---|---|---|---|
| | | Standard | | Standard | Water |
| Film former | Mean | deviation | Mean | deviation | resistance |
| Without film former | 22.7 | 2.9 | 19.9 | 3.1 | 87% |
| 3% Unimer U-151 | 21.3 | 0.6 | 20.4 | 1.6 | 96% |
| 3% (Boswellia | 25.6 | 0.2 | 24.0 | 0.3 | 94% |
| Thick Oil + | | | | | |
| Sweoat ® Oil PL40 | | | | | |
| FG) (40:60) | | | | | |

TABLE 8
Film formers in sunscreen with SPF 30
(synthetic and mineral UV filters)

| | SPF before bath | | SPF after bath | | |
|---|---|---|---|---|---|
| | | Standard | | Standard | Water |
| Film former | Mean | deviation | Mean | deviation | resistance |
| Without film | 27.3 | 5.7 | 19.9 | 3.9 | 73% |
| former | | | | | |
| 3% (Boswellia | 24.9 | 0.5 | 26.6 | 0.1 | 106% |
| Thick Oil + | | | | | |
| Sweoat ® Oil | | | | | |
| PL40 FG) | | | | | |
| (40:60) | | | | | |

1. Cosmetic ingredient comprising Boswellia thick oil and oat kernel oil in a ratio of between 60:40 and 15:85 by weight.

2. Cosmetic ingredient according to claim 1, comprising Boswellia thick oil and oat kernel oil in a ratio of between 50:50 and 20:80 by weight.

3. Cosmetic ingredient according to claim 1 consisting of Boswellia thick oil and oat kernel oil.

4. Cosmetic ingredient according to claim 1, wherein the oat kernel oil has a polar lipid content of at least about 25% by weight.

5. Cosmetic composition comprising the cosmetic ingredient according to claim 1, and a cosmetic active.

6. Cosmetic composition according to claim 5, further comprising a cosmetically acceptable excipient.

7. Cosmetic composition according to claim 6, wherein the cosmetically acceptable excipient is selected from the group consisting of: an oil-in-water emulsion, a water-in-oil emulsion, a lotion, a cream, an oil, a biphasic base, and an alcoholic base.

8. Cosmetic composition according to claim 5, comprising the cosmetic ingredient in an amount of about 1% to about 5%.

9. Cosmetic composition according to claim 5, wherein the cosmetic composition is a skin or hair care position composition.

10. Cosmetic composition according to claim 5, wherein the cosmetic active is selected from the group consisting of a UV filter, an anti-aging active, an antioxidant, a moisturizing active, a soothing active, and mixtures thereof.

11. Cosmetic composition according to claim 10, wherein the UV filter is selected from the group consisting of: bis-ethylhexyloxyphenol methoxyphenyl triazine, butyl methoxydibenzoylmethane, diethylamino hydroxybenzoyl hexyl benzoate, disodium phenyl dibenzimidazole tetrasulfonate, drometrizole trisiloxane, menthyl anthranilate, methylene bis-benzotriazolyl tetramethylbutylphenol, terephthalylidene dicamphor sulfonic acid, zinc oxide, 4-methylbenzylidene camphor, benzophenone-3, benzophenone-4, diethylhexyl butamido triazone, ethylhexyl methoxycinnamate, ethylhexyl salicylate, ethylhexyl triazone, ethylhexyl dimethyl para-aminobenzoic acid, homomenthyl salicylate, isoamyl p-methoxycinnamate, octocrylene, polyoxyethylen(25) para-aminobenzoic acid, phenylbenzimidazol sulfonic acid, Polysilicone-15, titanium dioxide, tris biphenyl triazine, and mixtures thereof.

12. Method of improving the water resistance of a cosmetic composition comprising the step of incorporating the cosmetic ingredient according to claim 1, in the cosmetic composition.

13. A film forming agent comprising cosmetic ingredient of claim 1.

14. Method of improving the water resistance of a cosmetic composition, comprising the step of using the cosmetic ingredient of claim 1, in the cosmetic composition.

15. Cosmetic ingredient according to claim 2, consisting of Boswellia thick oil and oat kernel oil in a ratio of between 45:55 and 25:75 by weight.

16. Cosmetic ingredient according to claim 2, comprising Boswellia thick oil and oat kernel oil in a ratio of between 45:55 and 35:65 by weight.

17. Cosmetic ingredient according to claim 4 wherein the oat kernel oil has a polar lipid content of at least about 30% by weight.

18. Cosmetic composition according to claim 5, comprising the cosmetic ingredient in an amount of about 2% to about 4%.

19. Cosmetic composition according to claim 5, wherein the cosmetic composition is a sun protection skin or hair care composition.

20. Cosmetic composition according to claim 11, wherein the UV filter is a sun filter selected from the group consisting of: bis-ethylhexyloxyphenol methoxyphenyl triazine, butyl methoxydibenzoylmethane, zinc oxide, ethylhexyl methoxycinnamate, ethylhexyl salicylate, ethylhexyl dimethyl para-aminobenzoic acid, homomenthyl salicylate, octocrylene, titanium dioxide, and mixtures thereof.
